(19) United States
(12) Patent Application Publication
(10) Pub. No.: US 2010/0257632 A1
(43) Pub. Date: Oct. 7, 2010

(54) METHODS FOR GENERATING MARKER-FREE TRANSGENIC PLANTS

(75) Inventors: Ilona Margaretha Bruggeman, Wageningen (NL); Michiel Theodoor Jan De Both, Wageningen (NL)

Correspondence Address: BROWDY AND NEIMARK, P.L.L.C.;624 NINTH STREET, NW, SUITE 300, WASHINGTON, DC, 20001-5303, US

(73) Assignee: KEYGENE N.V., Wageningen (NL)

(21) Appl. No.: 12/746,258

(22) PCT Filed: Dec. 5, 2008
(86) PCT No.: PCT/NL08/50775

§ 371 Date: Jun. 4, 2010

(30) Foreign Application Priority Data
Dec. 5, 2007 (EP) 071223549.9

(51) Int. Cl. C12N 15/82; A01H 1/00; C12N 1/21

(52) U.S. Cl. 800/266; 435/469; 435/252.2

(57) ABSTRACT

The invention relates to the field of plant transformation using Agrobacterium. An ultra-high co-transformation method is provided herein.

### FIELD OF THE INVENTION

The present invention relates to the field of plant transformation and methods for generating transgenic plant cells and plants and for generating marker free transgenic plants. Provided are methods for efficient co-transformation of plant cells using Agrbobacterium tumefaciens or Agrobacterium rhizogenes strains. Also provided are Agrobacterium strains suitable for use in the methods.

### BACKGROUND ART

Genetically modified, or “transgenic”, plants have been developed since the 1980s for various purposes, such as resistance to insect pests, herbicides or harsh environmental conditions (so-called “input traits”, benefiting the farmers during crop production), but also improved nutritional or health value (such as the golden rice, low linolenic acid soybean, etc.) (so-called “output traits”, benefiting the consumers and the processing industry). Despite debates about safety, new transgenic plants are being developed and it is expected that transgenic plant products with new, value added traits will reach the market in the coming years.

The use of a selectable marker gene is indispensable for the creation of genetically modified plants in order to select plant cells into which foreign DNA has been introduced, but after GM plants have been regenerated the selectable marker gene (generally an antibiotic resistance gene or a herbicide resistance gene) has become obsolete. The continued presence of a selectable marker gene in the GM plants is undesirable for several reasons. Public debate about safety of genetically modified organisms (GMOs) has led to guidelines stating that antibiotic resistance selectable marker genes should be preferably absent from GMOs for approval (EU directive 2001/18 EC). The prospect of renewed genetic transformation with additional genes of interest (GOI) of an existing GMO also requires the absence of selectable marker genes (or the use of alternative marker genes, which however leads to gene stacking), thus allowing a new round of selection in a new transformation cycle with one and the same selection marker.

The most straight-forward approach to eliminate selection markers is to apply a method called co-transformation, in which the selection marker used to select the transformed plant cells and the gene of interest (GOI) are placed on two distinct T-DNAs (usually in two Agrobacterium strains). The two T-DNAs are simultaneously introduced into a plant cell, but integrate independently from each other in different loci in the plant nuclear genome. Upon crossing, both genes subsequently segregate in the following generation, and GM plants with only the GOI can be selected. However, in this process also many plants are obtained with only the selectable marker gene, as the gene of interest is not selected for during the plant transformation and regeneration process. The challenge in co-transformation is obtaining a high percentage of transformed plant cells containing both genes (i.e. the same plant cell being “co-transformed” with both genes). With existing co-transformation methods, the percentage of plant cells containing both genes, i.e. the co-transformation efficiency, is low, as many cells receive only the T-DNA with the marker gene and not the T-DNA with the GOI. The efficiency lies at 50% or less. See also FIG. 4A.

For more detailed information on co-transformation see also De Block et al. (1991) Theoretical Applied Genetics 82: 257-263) Depicker et al. (1985) Molecular General Genetics 201: 477-484; Matthews et al. (2001) Mol. Breeding 7:195-202; Daley et al. (1998) PlantCell Rep. 17:489-496; McKnight et al (1987) Plant Molecular Biol. 8:439-445; and De Framond et al. (1986) Mol. Gen. Genet. 202:125-131.

Some improvements of co-transformation efficiencies of Agrobacterium-mediated transformation have been described. For example Japan Tobacco developed a specific approach to apply the co-transformation principle, using ‘superbinary’ vectors, and obtained 47% co-transformed plants in tobacco and rice (Komari et al., 1996, Plant J. 10-165-174; U.S. Pat. No. 5,731,179). Researchers at the University of Gent (De Buck et al., 1998, Mol. Plant. Microbe Interact 11: 449-457) reported a maximum of 50% co-transformation in Arabidopsis, but also found frequent co-integration of both T-DNAs at the same locus. In all these cases, co-transformation was performed by applying two functional Agrobacterium strains simultaneously, i.e. wherein each strain can transfer the T-DNA into the plant cell and facilitates transfer and integration into the nuclear plant genome when used alone. However, the overall efficiency remains low or unpredictable, making the method laborious and costly. Commonly, therefore, other transformation methods are used in the art, such as traditional methods where the selectable marker and GOI are physically linked on a T-DNA and are transferred into the plant cells on one piece of T-DNA. All transformed cells selected for having the marker gene therefore also contain the GOI, but removal of the marker gene is more difficult. The marker gene is then subsequently removed (post-transfomation) from the plant genome by excision using site specific recombination (using e.g. Cre-lox or FLP-frt systems). Other alternatives are for example the use of transposable elements or transformation with no selection marker at all (EP1279737). See also De Vetten et al. (2003) Nature Biotechnol. 21:439-442.

There remains, therefore, a need for a co-transformation method which has a high co-transformation efficiency, ideally approaching 100%. Such an ultra-high efficiency co-transformation method is provided herein, as are strains suitable for use in the method.

Agrobacterium strains contain a Ti-plasmid which comprises an opine synthesis and an opine catabolism region, a virulence region comprising genes encoding Vir proteins and a T-DNA region. During Agrobacterium mediated plant transformation, wound released chemicals, such as phenolic compounds and sugars, are recognized by the VirA transmembrane protein of Agrobacterium. Then, the VirA protein phosphorelates the VirG protein, which activates transcription of the other virulence genes of the vir region.

The VirD1 and VirD2 proteins cleave the borders and VirD2 remains covalently bound to the Right Border (RB). The VirB and VirD4 proteins form the pili for transport of the T-DNA/VirD2 complex into the plant cell. The VirE1 protein binds to the VirE2 protein and are transported together, separately from the T-complex, to the plant cytoplasm, where the VirE1 protein releases the VirE2 protein. The VirE2 protein then coats the single stranded (ss) T-DNA to protect the T-DNA against nucleases. The now complete T-complex will be transported to the nucleus, where VirE2 forms a channel in the nuclear membrane to accommodate import of the T-complex into the nucleus. In the nucleus, VirD2 is involved in T-DNA integration in the plant genome. The process described above is illustrated in FIG. 3 (Zhu et al, 2000, Journal of Bacteriology 182(14):3885-3895).

### DEFINITIONS

“Transformation” and “transformed” refers to the transfer of a DNA, generally a DNA comprising a chimeric gene of interest (GOI), into the nuclear genome of a plant cell to create a “transgenic” plant cell and plant comprising a transgene. The creation of so-called “cis-genic” plants by so-called “cis-genesis”, in which only DNA sequences from the host plant itself are being introduced, should also be understood to be “transformation”. The introduced DNA is generally, but not always, integrated in the host plant genome. Situations in which no DNA integration occur are for example the use of inverted repeat constructs to generate double-stranded RNA for gene silencing, or the use of DNA sequences coding for zinc-finger nucleases or other DNA modifying enzymes that are temporarily administered to the plant cell to obtain a permanent effect.

“Transfection” and “transfect” is used to refer to the T-DNA transfer into the plant cell, the step preceding transfer from the plant cytoplasm into the nucleus.

“Co-transformation” refers herein to the simultaneous transformation of a plant cell with two separate T-DNAs, one comprising a GOI and the other comprising a selectable marker gene. According to the present invention the two T-DNAs are present in two separate Agrobacterium strains.

“Co-transformation efficiency” refers to the percentage (number %) of regenerated transformed plants (transformants) selected for, using the marker gene, and comprising both of the two T-DNAs integrated in the nuclear genome.

“(Selectable) marker free plant” or “marker free transgenic plant” refers herein to a transgenic plant comprising a GOI integrated the nuclear genome of the cells, but lacking the plant selectable (or scorable) marker gene, which has been segregated away in the offspring of the transformants. “Offspring” or “descendents” or “progeny” may be the first or further generation obtained by selfing or crossing and which retain the chimeric gene, i.e. the GOI.

“T-DNA” (or “Transfer-DNA”) or “artificial or chimeric T-DNA” refers to a single or double stranded DNA comprising a right border (RB) and a left border (LB) sequence at either end or, in case of artificial T-DNA which is used to transfer a GOI at least a RB sequence. The “natural”, endogenous T-DNA region found in Agrobacterium Ti-plasmids is also referred to as T-DNA, but contains genes for tumor induction between the right and left borders. For plant transformation “disarmed” Agrobacterium strains are used, wherein these tumor inducing genes (tms and tmr regions) have been deleted, replaced or rendered non-functional. A T-DNA which is to be transferred into the plant cell is then introduced into the disarmed strain, generally on a plasmid or other vector, e.g. on a binary vector which does not integrate into the Ti-plasmid or on a co-integrate vector which integrates into the (disarmed) Ti-plasmid by homologous recombination. Agrobacterium strains and Ti-plasmids can be classified into different types based on the opine genes of the Ti-plasmid. The Ti-plasmid may contain opine synthesis genes (on the T-DNA region) and/or opine catabolism genes (on the Ti-plasmid backbone) such as octopine, nopaline or succinamopine synthesis and/or catabolism genes. Thus, different “opine type” Ti-plasmids exist.

“virE2 helper plasmid” in the context of the invention refers to a plasmid which can be introduced into the Agrobacterium strain and which comprises at least one virE2 gene and/or at least one complete virE operon of an Agrobacterium Ti-plasmid and which this produces a functional virE2 protein.

“Gene of interest” (GOI) refers to the chimeric gene which is to be integrated into the nuclear genome of a plant cell. A GOI may encode a protein of interest or a gene silencing construct.

“virE2 donor strain” refers to an Agrobacterium strain which is capable of producing functional virE2 protein and into which a T-DNA comprising a GOI is or can be introduced.

“virE2 mutant strain” refers to an Agorbobacterium strain which is not capable of producing functional virE2 protein and into which a T-DNA comprising a selectable marker gene is or can be introduced. The strain itself is avirulent (incapable of generating a transformed plant cell) when used alone, but is capable of producing a transformed plant cell when used together with a (virulent) virE2 donor strain. The endogenous virE2 gene on the Ti-plasmid is thus modified to not produce a functional virE2 protein.

“Plasmid” and “vector” are used herein interchangeably to refer to DNA molecules which may contain certain functions, such as an origin of replication (ori) functional in Agrobacterium and/or other bacteria used for DNA manipulation and replication (e.g. E. coli), one or more genes of interest, marker genes for selection in bacterial hosts or plant hosts, etc. Commonly known plasmids used in plant transformation are binary plasmids (which do not integrate into the Ti-plasmid and contain the T-DNA to be transferred into the plant), super-binary vectors, helper plasmids, Ti plasmids, Ti helper plasmids, etc.

“Ti plasmid” refers to natural Ti plasmids found in Agrobacterium species, such as A. tumefaciens or A. rhizogenes (in the latter they are called Ri plasmids, but for simplicity we herein use Ti plasmids to refer to both types of plasmids) or Ti plasmids derived therefrom and commonly used in plant transformation, such as “disarmed” Ti plasmids, which are not oncogenic (lacks functional tumor inducing genes of the T-DNA), but comprise the vir region of the Ti plasmid. See Hooykaas and Beijersbergen for a general review (Ann Rev Phytopathol 1994, 32: 157-179 or WO00/18939). Thus, natural Ti plasmids are large circular DNA molecules found in Agrobacterium strains, which comprise a virulence (vir) region and a T-DNA region (the T-DNA region being flanked by RB and LB sequences and comprising a tumor inducing region, with genes leading to auxin and cytokinin production and tumorigenesis, and an opine synthesis region next to the tumor inducing region), and further an opine catabolism region, an origin of replication and a conjugative transfer region.

An Agrobacterium strain “cured” for its endogenous Ti-plasmid is a strain wherein the own Ti-plasmid has been removed and into which a different Ti-plasmid can be introduced.

“LB” or “left border” and “RB” or “right border” sequences or “T-DNA border” sequences are short nucleotide sequences of about 25 by which flank the T-DNA region of Ti-plasmids and define the end points of DNA which is transferred from Agrobacterium into the plant cell. Border sequences are described in Gielen et al. (1984, EMBO J. 3, 835-845). RB and LB sequences can be added to the artificial T-DNAs comprising the marker gene or GOI, for example a plasmid may comprise, operably linked, the following DNA sequences: RB—gene (e.g. marker gene or GOI)—LB (optional), which may be inserted into a plasmid and into an Agrobacterium strain for transformation of a plant cell or plant.

The term “nucleic acid sequence” (or nucleic acid molecule) refers to a DNA or RNA molecule in single or double stranded form.

An “isolated nucleic acid sequence” refers to a nucleic acid sequence which is no longer in the natural environment from which it was isolated, e.g. the nucleic acid sequence in a bacterial host cell or in the plant nuclear genome.

The terms “protein” or “polypeptide” are used interchangeably and refer to molecules consisting of a chain of amino acids, without reference to a specific mode of action, size, 3-dimensional structure or origin. A “fragment” or “portion” of a protein may thus still be referred to as a “protein”. An “isolated protein” is used to refer to a protein which is no longer in its natural environment, for example in vitro or in a recombinant bacterial or plant host cell.

The term “gene” means a DNA sequence comprising a region (transcribed region), which is transcribed into an RNA molecule (e.g. an mRNA) in a cell, operably linked to suitable transcription regulatory regions (e.g. a promoter). A gene may thus comprise several operably linked sequences, such as a promoter, a 5′ non-translated leader sequence (also referred to as 5′UTR, which corresponds to the transcribed mRNA sequence upstream of the translation start codon) comprising e.g. sequences involved in translation initiation, a (protein) coding region (cDNA or genomic DNA) and a 3′ non-translated sequence (also referred to as 3′ untranslated region, or 3′UTR) comprising e.g. transcription termination sites and polyadenylation site (such as e.g. AAUAAA or variants thereof).

A “chimeric gene” (or recombinant gene) refers to any gene, which is not normally found in nature in a species, in particular a gene in which one or more parts of the nucleic acid sequence are present that are not associated with each other in nature. For example the promoter is not associated in nature with part or all of the transcribed region or with another regulatory region. The term “chimeric gene” is understood to include expression constructs in which a promoter or transcription regulatory sequence is operably linked to one or more sense sequences (e.g. coding sequences) or to an antisense (reverse complement of the sense strand) or inverted repeat sequence (sense and antisense, whereby the RNA transcript forms double stranded RNA upon transcription).

“cis-genesis” and “cis-gene” refers to the generation of transgenic plants or plant cellswith genes composed of genetic elements of the plant species which is being transformed or of a closely related (sexually compatible) species. A cis-gene includes its introns and is flanked by its native promoter and terminator in the normal sense orientation. Cis-genic plants can harbour one or more ci-sgenes, but they do not contain any chimeric genes. Throughout the description it is clear that instead of or in addition to using chimeric genes also cis-genes can be used and this embodiment is encompassed herein throughout.

“In trans” refers to being present on separate DNA molecules, while “in cis” refers to being present on the same DNA molecule. For example the Agrobacterium vir genes can be present in the Agrobacterium strain in trans in relation to the T-DNA to be transferred, while the T-DNA border sequences are in cis in relation to the gene of interest which they flank.

A “3′ UTR” or “3′ non-translated sequence” (also often referred to as 3′ untranslated region, or 3′ end) refers to the nucleic acid sequence found downstream of the coding sequence of a gene, which comprises, for example, a transcription termination site and (in most, but not all eukaryotic mRNAs) a polyadenylation signal (such as e.g. AAUAAA or variants thereof). After termination of transcription, the mRNA transcript may be cleaved downstream of the polyadenylation signal and a poly(A) tail may be added, which is involved in the transport of the mRNA to the cytoplasm (where translation takes place).

“Expression of a gene” refers to the process wherein a DNA region, which is operably linked to appropriate regulatory regions, particularly a promoter, is transcribed into an RNA, which is biologically active, i.e. which is capable of being translated into a biologically active protein or peptide (or active peptide fragment) or which is active itself (e.g. in posttranscriptional gene silencing or RNAi). An active protein in certain embodiments refers to a protein having a dominant-negative function due to a repressor domain being present. The coding sequence is preferably in sense-orientation and encodes a desired, biologically active protein or peptide, or an active peptide fragment (i.e. the protein or peptide encoded by the GOI). In gene silencing approaches, the DNA sequence is preferably present in the form of an antisense DNA or an inverted repeat DNA, comprising a short sequence of the target gene in antisense, or in sense and antisense orientation (the sense and antisense RNA can hybridize with each other to form a dsRNA or stem-loop structure). “Ectopic expression” refers to expression in a tissue in which the gene is normally not expressed.

A “transcription regulatory sequence” is herein defined as a nucleic acid sequence that is capable of regulating the rate of transcription of a nucleic acid sequence operably linked to the transcription regulatory sequence. A transcription regulatory sequence as herein defined will thus comprise all of the sequence elements necessary for initiation of transcription (promoter elements), for maintaining and for regulating transcription, including e.g. attenuators or enhancers, but also silencers. Although mostly the upstream (5′) transcription regulatory sequences of a coding sequence are referred to, regulatory sequences found downstream (3′) of a coding sequence are also encompassed by this definition.

As used herein, the term “promoter” refers to a nucleic acid fragment that functions to control the transcription of one or more genes, located upstream (5′) with respect to the direction of transcription of the transcription initiation site of the gene (the transcription start is referred to as position +1 of the sequence and any upstream nucleotides relative thereto are referred to using negative numbers), and is structurally identified by the presence of a binding site for DNA-dependent RNA polymerase, transcription initiation sites and any other DNA domains (cis acting sequences), including, but not limited to transcription factor binding sites, repressor and activator protein binding sites, and any other sequences of nucleotides known to one of skill in the art to act directly or indirectly to regulate the amount of transcription from the promoter. Examples of eukaryotic cis acting sequences upstream of the transcription start (+1) include the TATA box (commonly at approximately position −20 to −30 of the transcription start), the CAAT box (commonly at approximately position −75 relative to the transcription start), 5′ enhancer or silencer elements, etc. A “constitutive” promoter is a promoter that is active in most tissues (or organs) under most physiological and developmental conditions. More preferably, a constitutive promoter is active under essentially all physiological and developmental conditions in all major organs, such as at least the leaves, stems, roots, seeds, fruits and flowers. Most preferably, the promoter is active in all organs under most (preferably all) physiological and developmental conditions.

An “inducible” promoter is a promoter that is physiologically (e.g. by external application of certain compounds) or developmentally regulated. A “tissue specific” promoter is only active in specific types of tissues or cells.

As used herein, the term “operably linked” refers to a linkage of polynucleotide elements in a functional relationship. A nucleic acid is “operably linked” when it is placed into a functional relationship with another nucleic acid sequence. For instance, a promoter, or a transcription regulatory sequence, is operably linked to a coding sequence if it affects the transcription of the coding sequence. Operably linked means that the DNA sequences being linked are typically contiguous and, where necessary to join two protein encoding regions, contiguous and in reading frame so as to produce a “chimeric protein”. A “chimeric protein” or “hybrid protein” is a protein composed of various protein “domains” (or motifs) which is not found as such in nature but which are joined to form a functional protein, which displays the functionality of the joined domains (for example a DNA binding domain or a repression of function domain leading to a dominant negative function). A chimeric protein may also be a fusion protein of two or more proteins occurring in nature. The term “domain” as used herein means any part(s) or domain(s) of the protein with a specific structure or function that can be transferred to another protein for providing a new hybrid protein with at least the functional characteristic of the domain.

The term “target peptide” refers to amino acid sequences which target a protein to intracellular organelles such as plastids, preferably chloroplasts, mitochondria, or to the extracellular space (secretion signal peptide). A nucleic acid sequence encoding a target peptide may be fused (in frame) to the nucleic acid sequence encoding the amino terminal end (N-terminal end) of the protein.

A “nucleic acid construct” or “vector” or “plasmid” is herein understood to mean a man-made (usually circular) nucleic acid molecule resulting from the use of recombinant DNA technology and which is used to deliver exogenous DNA into a host cell. The vector backbone may for example be a binary or superbinary vector (see e.g. U.S. Pat. No. 5,591,616, US2002138879 and WO 95/06722), a co-integrate vector (which integrates into the Ti-plasmid) or a T-DNA vector, as known in the art and as described elsewhere herein, into which a chimeric gene is integrated or, if a suitable transcription regulatory sequence/promoter is already present, only a desired nucleic acid sequence (e.g. a coding sequence, an antisense or an inverted repeat sequence) is integrated downstream of the transcription regulatory sequence/promoter. Vectors usually comprise further genetic elements to facilitate their use in molecular cloning, such as e.g. selectable markers, multiple cloning sites and the like (see below).

A “host cell” or a “recombinant host cell” or “transformed cell” are terms referring to a new individual cell (or organism), arising as a result of the introduction into said cell of at least one nucleic acid molecule, especially comprising a chimeric gene encoding a desired protein or a nucleic acid sequence which upon transcription yields an antisense RNA or an inverted repeat RNA (or hairpin RNA) for silencing of a target gene/gene family. The host cell is preferably a plant cell, but may also be a bacterial cell (e.g. an Agrobacterium strain), a fungal cell (including a yeast cell), etc. The host cell may contain the nucleic acid construct as an extra-chromosomally (episomal) replicating molecule, or comprises the chimeric gene integrated in the nuclear of the host cell.

The term “selectable marker” or “scorable marker” is a term familiar to one of ordinary skill in the art and is used herein to describe any genetic entity which, when expressed, can be used to select for a cell or cells containing the selectable marker, e.g. a “plant selectable marker gene” can be used to select plant cells comprising the gene. Selectable marker gene products confer, for example, antibiotic resistance, or more preferably, herbicide resistance or another selectable trait such as a phenotypic trait (e.g. a change in pigmentation) or a nutritional requirement. The term “reporter” is mainly used to refer to visible markers, such as green fluorescent protein (GFP), eGFP, luciferase, GUS and the like.

The terms “homologous” and “heterologous” refer to the relationship between a nucleic acid or amino acid sequence and its host cell or organism, especially in the context of transgenic organisms. A homologous sequence is thus naturally found in the host species (e.g. a tomato plant transformed with a tomato gene), while a heterologous sequence is not naturally found in the host cell (e.g. a tomato plant transformed with a sequence from potato plants).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. It is further understood that, when referring to “sequences” herein, generally the actual physical molecules with a certain sequence of subunits (e.g. nucleic acids or amino acids) are referred to.

Whenever reference to a “plant” or “plants” (or a plurality of plants) according to the invention is made, it is understood that also plant parts (cells, tissues or organs, seeds, severed or harvested parts, leaves, seedlings, flowers, pollen, fruit, stems, roots, callus, protoplasts, etc), progeny or clonal propagations of the plants which retain the distinguishing characteristics of the parents (e.g. presence of a trans-gene), such as seed obtained by selfing and/or crossing, e.g. hybrid seed (obtained by crossing two inbred parental lines), hybrid plants and plant parts derived therefrom are encompassed herein, unless otherwise indicated.

The term “substantially identical”, “substantial identity” or “essentially similar” or “essential similarity” or “variant” means that two peptide or two nucleotide sequences, when optimally aligned, such as by the programs GAP or BESTFIT using default parameters, share at least a certain percent sequence identity. GAP uses the Needleman and Wunsch global alignment algorithm to align two sequences over their entire length, maximizing the number of matches and minimizes the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). It is clear that when RNA sequences are said to be essentially similar or have a certain degree of sequence identity with DNA sequences, thymine (T) in the DNA sequence is considered equal to uracil (U) in the RNA sequence. Sequence alignments and scores for percentage sequence identity may be determined using computer programs, such as the GCG Wisconsin Package, Version 10.3, available from Accelrys Inc., 9685 Scranton Road, San Diego, Calif. 92121-3752 USA. or using in EmbossWIN (version 2.10.0) the program “needle”, using the same GAP parameters as described above or using gap opening penalty 10.0 and gap extension penalty 0.5, using DNAFULL as matrix. For comparing sequence identity between sequences of dissimilar lengths, it is preferred that local alignment algorithms are used, such as the Smith Waterman algorithm (Smith TF, Waterman Miss. (1981) J. Mol. Biol. 147(1); 195-7), used e.g. in the EmbossWIN program “water”. Default parameters are gap opening penalty 10.0 and gap extension penalty 0.5, using Blosum62 for proteins and DNAFULL matrices for nucleic acids.

### DETAILED DESCRIPTION OF THE INVENTION

The instant invention provides an ultrahigh efficient co-transformation method, having a co-transformation efficiency of above 50%, especially equal to or above 60%, 70%, 80%, 90% and in particular having a co-transformation efficiency of about 100%.

It was found that the use of two Agrobacterium strains, one comprising a gene for a functional virE2 protein (the virE2 donor strain) and one strain not capable of producing a functional virE2 protein (the virE2 mutant strain) can be used to achieve very high co-transformation efficiencies. The virE2 mutant strain used alone is able to transport its own T-DNA strand with the selectable marker gene into the plant cell (i.e. transfects the cell), but it cannot process the T-DNA further and cannot, therefore, integrate the T-DNA into the nuclear genome of the plant cell, i.e. no transformed plant cell and plant can be made with the selectable marker gene.

When a virE2 donor strain is used together in co-inoculations with the virE2 mutant strain, the virE2 protein and T-DNA (comprising the GOI) of the donor strain is transferred into the plant cell. The virE2 protein produced by the donor strain enters the plant cells and associates to both the T-DNA comprising the GOI and the T-DNA comprising the selectable marker gene (introduced from the virE2 mutant strain) and thereby enables the integration of both T-DNAs into the nuclear genome of the same plant cell. Thus, only those plant cells which have been co-inoculated with both the virE2 donor strain and the virE2 mutant strain will be able to integrate both T-DNAs into the nuclear genome. Selection for the phenotype conferred by the selectable marker gene being expressed results in at least about 60% or more (up to 100%) of the selected transformants comprising both the marker gene and the GOI integrated into the nuclear genome of the transformants. Because the separate T-DNAs integrate at random locations in the genome they are inherited independently and subsequent segregation of the GOI and the marker gene occurs in the offspring of the transformants, enabling the marker gene to be selected away from the GOI (generating marker free plants comprising only the gene-of-interest, and not the selectable marker gene).

Thus, in one embodiment a method for making (marker free) transgenic plants comprising a gene of interest is provided, the method comprising the steps of:
a) providing two Agrobacterium strains, a virE2 donor strain comprising a gene encoding a functional virE2 protein and a virE2 mutant strain not capable of producing a functional virE2 protein,
b) introducing a T-DNA comprising a gene-of-interest into the virE2 donor strain,
c) introducing a T-DNA comprising a selectable marker gene into the virE2 mutant strain,
d) contacting (e.g. co-inoculating or co-infecting) plant cells with both strains, and
e) selecting plant cells and/or regenerated plants (or plantlets) using the phenotype conferred by the selectable marker gene product, and optionally
f) crossing and/or selfing the selected plants (or plants derived from the selected plant cells or plantlets) to produce offspring, and optionally
g) discarding those offspring which comprise the selectable marker gene and retaining those offspring which comprise the gene of interest but lack the selectable marker gene (i.e. segregating away the marker gene from the gene of interest to produce marker free plants comprising the gene of interest).

Any plant host which can be transformed with Agrobacterium strains can be transformed according to the invention, i.e. monocotyledonous or dicotyledonous species, using methods known in the art, in combination with the two Agrobobacterium strains according to the invention. See for example Fraley et al. (1983, Proc Natl Acad Sci USA 80:4803; Comai et al. (1994, Nature 317:741), Shah et al. (1986, Science 233: 478) and others for Agrobacterium mediated gene transfer. The use of Agrobacterium to transform plant cells, and thereafter, regenerate a transformed plant from the transformed plant cell uses procedures described, for example, in EP 0 116 718, EP 0 270 822, PCT publication WO 84/02913 and published European Patent application EP 0 242 246 and in Gould et al. (1991, Plant Physiol. 95, 426-434). The construction of a T-DNA vector for Agrobacterium mediated plant transformation is well known in the art. The T-DNA vector may be either a binary vector as described in EP 0 120 561 and EP 0 120 515 or a co-integrate vector which can integrate into the Agrobacterium Ti-plasmid by homologous recombination, as described in EP 0 116 718.

Step (a) of the method involves providing two Agrobacterium strains, a virE2 donor strain comprising a gene encoding a functional virE2 protein (virE2⁺ strain) and a virE2 mutant strain not capable of producing a functional virE2 protein (virE2⁰ strain). When plant cells are co-infected with both strains, the virE2⁺ strain is able to complement the virE2⁰ strain and the virE2 protein supplied by the one strain will lead to integration of both T-DNAs (the T-DNA transfected from the first strain and from the second strain) into the nuclear genome of the transfected plant cell. The fact that the virE2 protein can enter plant cells independent of the T-DNA strand has already been shown in the art by complementation experiments, whereby co-infection with two Agrobacterium strains—one lacking T-DNA but containing functional virE2 and the other lacking virE2 but containing T-DNA—lead to plant cells containing the T-DNA integrated in the genome (Otten et al. 1984 Mol Gen Genet. 175, 159-163; Dombek and Ream J of Bacteriol 1997, Vol. 79, 1165-1173). However, up to date it was not known that this finding could be exploited for improving the co-transformation efficiency of plant cell significantly and nobody has introduced a T-DNA comprising a plant selectable marker gene into a virE2⁰ strain and a T-DNA comprising a GOI into a virE2⁺ strain and used these two strains together in co-infections of plant cells to generate marker free plants. In Dombek and Ream (1997, supra) the strain supplying the functional virE2 protein contains no T-DNA and comprises also no gene-of-interest, as the idea therein is to test whether the virE2 protein functionally complements the virE2 mutation in the other null strain in order to study the role of virE2 in nature. Also, the mutant/null strain used is oncogenic and not disarmed, leading to tumorigenesis in case of functional complementation. In the instant invention preferably disarmed Ti-plasmids and thus disarmed Agrobacterium strains are used.

Further, it was surprisingly found by the instant inventors that the use of at least one further virE2 gene in the donor strain appears to provide an amount of virE2 protein which is sufficient for associating with the two different T-DNAs and for transferring these into the nuclear genome of the same plant cell. Thus, in a preferred embodiment of the invention the virE2 donor strain comprises at least two virE2 genes, each encoding a functional virE2 protein. The virE2 genes may be integrated into the genomic DNA, Ti-plasmid and/or further plasmid within the strain and need not to (although they may) be present on the same genetic element.

Optionally, the virE2 donor strain also comprises a further virG gene, encoding for a functional virG protein. Thus, in one embodiment the strain comprises at least two virE2 genes and at least two virG genes, all encoding functional proteins. The genes may be transcribed from native promoters (i.e. native virE2 and virG promoters) or from different promoters functional in Agrobacterium, such as inducible promoters, constitutive promoters, promoters from virE2 or virG genes of other Agrobacterium strains, etc. Agrobacterium tumefaciens VirG nucleic acid and protein sequences from various strains are available in the art, see e.g. Schrammeijer (Journal of Experimental Botany, Vol. 51, No. 347, pp. 1167-1169, Jun. 2000) or GenBank Accession number X62885 (SEQ ID NO: 4), NP 059810, AAA91602 and others. VirG proteins comprise thus, for example, at least 80%, 90%, 95%, 98%, 99% or more amino acid identity when aligned with e.g. the protein of SEQ ID NO: 4 (X62885) or to the VirG of EHA105 (pTiBo542) (Chen, C. Y. et al. Mol. Gen. Genet. 230 (1-2), 302-309 (1991).

virE2 Donor Strains

Suitable virE2 donor strains include any Agrobacterium strain capable of infecting plant cells naturally, such as Agrobacterium tumefaciens strains and Agrobacterium rhizogenes strains. The strain preferably comprises a (preferably disarmed) Ti-plasmid which carries the natural (wild type) virE operon and thus the natural virE2 gene. Any Agrobacterium strain commonly used in plant transformation methods is suitable, as long as the strain is virulent. Preferably the strain is disarmed, i.e. the tmr and tms genes found on the natural T-DNA region of A. tumefaciens are removed or non-functional, so that no plant tumors develop after T-DNA transfer. The whole native T-DNA region of a natural Ti-plasmid may be removed to disarm the strain. The Ti-plasmid and/or Agrobacterium strain may be of any opine type, but in a preferred embodiment a succinamopine Ti-plasmid is used, which comprises the genes for succanimopine catabolism. Succinamopine Ti-plasmids are available in the art (e.g. strain EHA105). It is understood that (disarmed) Ti-plasmids can be manipulated in and outside of Agrobacterium and can be introduced or transferred into any Agrobacterium strain, for example a strain which is cured of its native Ti-plasmid. Likewise, other plasmids may be introduced into Agrobacterium strains, providing further functions, such as the T-DNA which is to be introduced into the plant cells, extra virE2 and/or virG genes, etc. In addition, genetic elements may be introduced into the genomic DNA of the strain. Thus, the genetic elements referred to herein, according to the invention, may be introduced into one or more different parts of an Agrobacterium strain, such as one or more Ti-plasmids, one or more further plasmids, the genomic DNA, etc. Even if this is not explicitly mentioned in the description, it is understood to be encompassed herein.

Alternatively or in addition to the virE2 gene found on the (preferably disarmed) Ti-plasmid, one or more further virE2 genes may be supplied in cis or trans with respect to the other vir genes found naturally on the Ti-plasmid of Agrobacterium strains and required for virulence. Thus, in one embodiment the Agrobacterium strain comprises one or more virE2 genes on the Ti-plasmid, genomic DNA and/or further plasmids.

For example, one or more plasmids (e.g. the Ti-plasmid and/or other plasmids) comprising a virE2 gene encoding a functional virE2 protein, operably linked to a suitable transcription regulatory region which is active or inducible in the strain, are introduced into the strain. The plasmid(s) may also comprise the whole virE operon found in an Agrobacterium Ti-plasmid. Preferably at least two genes encoding functional virE2 protein are present in the strain, but also more may be present, such as 3, 4, 5 or more. Disarmed Agrobacterium strains, comprising a Ti-plasmid and at least one virE2 gene encoding a functional virE2 protein are widely available in the art.

Additional virE2 genes can be introduced using known method, by e.g. operably linking the coding sequence of a virE2 gene to a promoter active in Agrobacterium (e.g. the natural virE2 promoter) and inserting the gene into a suitable plasmid and/or into the Ti-plasmid of the strain and/or into the genomic DNA. Additional virE2 genes may for example be inserted into the vir-region of the Ti-plasmid, or into another location of the Ti-plasmid. In one embodiment at least two virE2 genes are present in trans on one genetic element, e.g. the Ti-plasmid or another plasmid. In another embodiment at least two virE2 genes are present in cis, e.g. on different genetic elements. For example, one virE2 gene is on a Ti-plasmid and one virE2 gene is on a helper plasmid. The virE2 genes encode functional VirE2 proteins.

Further, the virE2 donor strain also comprises at least one, optionally at least 2, 3, 4 or more, virE1 genes, encoding a functional virE1 protein, which is employed in transporting the virE2 proteins into the plant cell. The virE1 gene may be present in the Agrbobacterium genome, the Ti-plasmid (i.e. the Ti-plasmid may comprise the native virE1 gene and promoter and/or one or more other virE1 genes and promoters may be inserted) and/or on another plasmid. Thus, the Ti-plasmid or other plasmid which is used to introduce the virE2 gene(s) may in one embodiment also comprise at least one virE1 gene, preferably operably linked to a promoter active in Agrobacterium, e.g. a virE operon promoter. Genes encoding Agrobacterium virE protein are available in the art, see e.g. GenBank accession AAZ50537 (SEQ ID NO: 5; virE1 of pTiBo152). “VirE1” includes variants, such as virE2 proteins comprising at least 80%, 90%, 95%, 98%, 99% or more amino acid sequence identity to SEQ ID NO: 5.

Preferably the strain is capable of making sufficient virE2 protein upon co-inoculation with a virE2 mutant strain to associate with both the single stranded T-DNA provided by the virE2 mutant strain (the marker gene comprising T-DNA) and the single stranded T-DNA provided by the virE2 donor strain (the GOI comprising T-DNA). Whether “sufficient” virE2 protein is being made can be determined by the skilled person using routine experimentation, for example if the co-transformation efficiency is lower than 50%, then there is likely insufficient virE2 protein being made and the levels need to be increased by e.g. adding one or more further virE2 genes (e.g. on a helper plasmid).

The virE2 gene has been cloned and can be introduced into any plasmid and/or Agrobacterium strain as desired. SEQ ID NO: 1 (cDNA) and SEQ ID NO: 2 (protein encoded by SEQ ID NO: 1) provide the virE2 genes/protein of Ti plasmid pTiBo542. VirE2 genes and proteins are also available in public databases, see AAZ50538 (virE2 from pTiBo542), NP 059819 and others.

SEQ ID NO: 3 provides the virE2 genes, with an insertion in the virE2 open reading frame. The insertion was generated by integration (homologous recombination) of a plasmid comprising a part of the virE2 open reading frame (ORF) into the native virE2 ORF. The insertion may occur at any position in the virE2 ORF and occurred herein after nucleotide 203 of SEQ ID NO: 1. Thus, the virE2 mutant strain deposited herein as CBS121809 comprises a Ti-plasmid with SEQ ID NO: 3 (comprising a disruption of SEQ ID NO: 1), leading to no virE2 protein being produced in vivo (see below).

Obviously, other virE2 sequences, encoding functional virE2 protein, can be made or identified (e.g. in silico) or isolated from Agrobacterium strains using known methods in the art. A virE2 gene according to the invention encompasses genes encoding essentially similar amino acid sequences (also referred to as “variants”), such as nucleotide sequences encoding a protein which comprises at least 70%, preferably at least 80%, 85%, 90%, 95%, 98%, 99% or more amino acid sequence identity to SEQ ID NO: 2 (using pairwise alignment over the entire length, for example using the program “needle” of EmbossWin with a Gap opening penalty of 10.0 and gap extension penalty 0.5, using Blosum62 as matrix). Thus, virE2 genes, encoding functional virE2 proteins or variants thereof (e.g. homologs) may be isolated from other organisms, especially other Agrobacterium strains and used to produce functional virE2 proteins in the virE2 donor strain. Other virE2 nucleic acid and/or amino acid sequences may also be identified in silico, in DNA and/or protein databases or by nucleic acid hybridization or PCR, using the virE2 sequence or part thereof as probe or primer. The same applies for virG and virE1 genes and proteins (and variants thereof) described further above.

Functionality of the encoded virE2 protein and/or variants can be easily tested, using e.g. complementation assays as described in the Examples.

Obviously, virE2 nucleic acid sequences according to the invention also encompass variants, such as nucleic acid sequence comprising at least 70%, preferably at least 80%, 85%, 90%, 95%, 98%, 99% or more nucleic acid sequence identity to SEQ ID NO: 1 (using pairwise alignment over the entire length, for example using the program “needle” of EmbossWin with a Gap opening penalty of 10.0 and gap extension penalty 0.5, using DNAfull as matrix).

virE2 Mutant Strains

The second strain used in the method is a virE2 mutant strain, not capable of producing a functional virE2 protein. When referring to “no functional virE2 being made” this includes the possibility that either no virE2 protein is/can be made at all by the strain or that non-functional protein is/can be made.

Again, any Agrobacterium strain may be used and any Ti-plasmid, as long as the endogenous virE2 gene found on the (preferably disarmed) Ti-plasmid is modified so that no functional protein is/can be made. VirE2 mutant strains can be made analogous to the way described in the Examples for EHA105-dE2 (CBS121809), by for example inserting a plasmid into the virE2 ORF by homologous recombination. Obviously, many other methods are available in the art to achieve the same result. Insertions, deletions and/or replacements of all or part of the virE2 gene (e.g. SEQ ID NO: 1 or variants thereof) on the Ti-plasmid will result in a virE2 mutant strain and in a Ti-plasmid comprising a mutant virE2 gene or lacking a virE2 gene. When referring to a “mutant” virE2 gene any genetic modification which results in substantially no functional virE2 protein being made is encompassed, i.e. mutations/genetic modification by insertion, replacement or deletion of all or part of the virE2-protein encoding DNA. Mutant virE2 genes may also be isolated or made synthetically or using molecular biology techniques and may then be used to replace a functional virE2 gene on a Ti-plasmid to be used in the strain.

The lack of virE2 activity can be determined as described in the Examples. Alternatively, other assays may be used as known.

In one embodiment of the invention the virE2 mutant is the strain deposited under Accession number CBS121809 (or a derivative thereof), or an Agrobacterium strain comprising the Ti-plasmid comprised therein, or the mutant virE2 gene comprised therein. The Ti-plasmid comprising the virE2 mutant gene may be transferred into another strain, e.g. a cured strain. Alternatively, the mutant gene may be isolated from the deposited strain and transferred into another strain and/or another Ti-plasmid.

Some other VirE2 mutant Agrobacterium strains have also already been described in the art, but these strains have not been used in co-transformation methods but only to study the function of virE2. For example Simone et al. 2001 (Mol Microbiol Vol 41: 1283-1293), Dombek and Ream (1997, J of Bacteriology Vol 179: 1165-1173), Binns et al. (1995, J of Bacteriol. Vol 177: 4890-4899) and Stachel and Nester (1986; EMBO J. Vol. 5: 1445-1454) describe the generation of virE2 mutants and virE2 mutant strains. The strains are however not disarmed and they are not used in co-transformation, i.e. the complementation assay makes use of a wild type strain to provide functional virE2, which does however not contain a T-DNA with a GOI. In addition, all of the virE2 mutant strains are octopine type strains.

The virE2 mutant strains described in the above references may also be used in the instant invention, although preferably the strains are disarmed prior to use. The strains include A. tumefaciens strain WR5000 containing pTiA6NC with virE2 replaced by nptII (Dombek and Ream, supra, see FIG. 1 therein) or another virE2 mutant derived by mutating the virE2 gene on the Ti-plasmid pTiA6NC (e.g. found in strain A348); A. tumefaciens strains as listed in Table 1 of Binnes et al. (supra), such as A348::virE2 in which a plasmid has been integrated into the ORF of virE2; Agrobacterium strains 358mx described in Stachel and Nester (supra), comprising a transposon insertion in the virE2 gene.

Basically, any Agrobacterium strain can be modified to comprise a mutation in the virE2 gene or to lack the virE2 gene. In one embodiment of the invention the virE2 mutant strain, and/or a virE2 mutant Ti-plasmid, is provided, wherein the mutant strain and/or Ti-plasmid is a succinamopine Ti-plasmid and/or a strain comprising such a plasmid. The virE2 mutant strains according to the invention are, thus, in one embodiment preferably succinamopine strains, e.g. carrying a Ti plasmid derived from pTiBo542 (EHA101, EHA105). These strains have a broad host range; meaning that they are capable to transforming many different plant host species.

The virE2 gene comprises an insertion, deletion or replacement of all or part of the virE2 gene, whereby the virE2 protein is not made or is nonfunctional (e.g. truncated or does not fold properly). In one embodiment the virE2 mutant strain is EHA105-dE2 deposited under Accession number CBS121809 and the Ti-plasmid is the plasmid present in this strain, or a derivative of any of these. Also, an isolated mutant virE2 DNA is provided as depicted in SEQ ID NO: 3, comprising an insertion (knock-out) in the virE2 cDNA of SEQ ID NO: 1. SEQ ID NO: 3 shows the whole VirE operon region of the mutant strain, including the plasmid that was inserted into the virE2 ORF, disrupting it in the process.

Thus, in one embodiment of the invention the virE2 mutant strain comprises one or more insertions, deletions and/or replacements in the virE2 gene, wherein part or all of the virE2 gene is deleted and/or replaced and/or wherein DNA is inserted, whereby the changes lead to the absence of any virE2 protein being made or to non-functional virE2 protein being made (e.g. truncated, non-functional protein).

In step (b) of the method a T-DNA comprising a gene-of-interest is introduced into the virE2 donor strain and in step (c) a T-DNA comprising a selectable marker gene is introduced into the virE2 mutant strain. Thus, suitable T-DNAs are provided, comprising for example operably linked elements such as RB and (optionally) LB, GOI or marker. The T-DNA can be made using standard molecular biology methods. Preferably the T-DNA is on a plasmid vector, such as a binary vector or co-integrate vector. Introduction of the T-DNA, or vector comprising the T-DNA, into the Agrobacterium strain can be done by e.g. electroporation.

Thus, in one embodiment of the method, the T-DNAs are introduced on a DNA vector or plasmid and the T-DNAs comprise at least a right border sequence.

The GOI may be any gene, whether encoding a protein or having a different function, such as (but not limited to) selected from: genes (coding sequences such as cDNA or genomic DNA) for biotic and/or abiotic stress tolerance, disease resistance, herbicide resistance, agronomic traits, output traits, etc. The GOI may be a plant or plant-derived gene, or alternatively a gene naturally found in bacteria, fungi, animals (including humans), etc. In one embodiment the gene is a cis-gene. Genomic DNA and cDNA of suitable coding sequences are available in the art, e.g. in nucleic acid databases.

Marker genes include plant selectable markers, such as (but not limited to) resistance genes (e.g. antibiotic resistance, herbicide resistance, etc.) or other genes conferring a trait which can be used to select transformants, such as those conferring a phenotypic trait.

In step (d) the plant, plant part, plant cell(s) or tissues to be transformed are contacted (e.g. co-inoculating or co-infecting) with a suitable amount and/or ratio of both strains (i.e. at least one virE2 donor strain and at least one virE2 mutant strain) under suitable conditions and for a suitable period of time, as for example described in established Agrobacterium transformation protocols or adapted therefrom. See Example 2 for tomato transformation references and protocol outline. Co-inoculation or co-infection refer herein to either both being physically added to the cells/tissue together (as a mixture or at the same time) or to the strains being added consecutively. In case of consecutive contact it is preferred to add the strain comprising the GOI first, as the GOI can (usually) not be selected for, followed by the strain comprising the selectable marker gene within a short time interval.

The plant cells or tissues may for example be explants of cotyledons or other tissues, as known in the art, such as root cultures, leaf discs, etc. Alternatively parts or all of a plant may be contacted, e.g. by infiltration. Protocols for Agrobacterium transformation are known in the art and may be used according to the invention. The plant cells, tissue or plant may be of any species which is amenable to Agrobacterium transformation. Thus, any plant may be suitable, such as monocotyledonous plants or dicotyledonous plants, for example maize/corn (Zea species, e.g. Z. mays, Z. diploperennis (chapule), Zea luxurians (Guatemalan teosinte), Zea mays subsp. huehuetenangensis (San Antonio Huista teosinte), Z. mays subsp. mexicana (Mexican teosinte), Z. mays subsp. parviglumis (Balsas teosinte), Z. perennis (perennial teosinte) and Z. ramosa, wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp (e.g. B. napus, B. juncea, B. oleracea, B. rapa, etc.), sunflower (Helianthus annus), tobacco (Nicotiana species), alfalfa (Medicago sativa), rice (Oryza species, e.g. O. sativa indica cultivar-group or japonica cultivar-group), forage grasses, pearl millet (Pennisetum species. e.g. P. glaucum), tree species, vegetable species, such as Lycopersicon ssp (recently reclassified as belonging to the genus Solanum), e.g. tomato (L. esculentum, syn. Solanum lycopersicum) such as e.g. cherry tomato, var. cerasiforme or current tomato, var. pimpinellifolium) or tree tomato (S. betaceum, syn. Cyphomandra betaceae), potato (Solanum tuberosum) and other Solanum species, such as eggplant (Solanum melongena), pepino (S. muricatum), cocona (S. sessiliflorum) and naranjilla (S. quitoense); peppers (Capsicum annuum, Capsicum frutescens), pea (e.g. Pisum sativum), bean (e.g. Phaseolus species), carrot (Daucus carona), Lactuca species (such as Lactuca sativa, Lactuca indica, Lactuca perennis), cucumber (Cucumis sativus), melon (Cucumis melo), zucchini (Cucurbita pepo), squash (Cucurbita maxima, Cucurbita pepo, Cucurbita mixta), pumpkin (Cucurbita pepo), watermelon (Citrullus lanatus syn. Citrullus vulgaris), fleshy fruit species (grapes, peaches, plums, strawberry, mango, melon), ornamental species (e.g. Rose, Petunia, Chrysanthemum, Lily, Tulip, Gerbera species), woody trees (e.g. species of Populus, Salix, Quercus, Eucalyptus), fibre species e.g. flax (Linum usitatissimum) and hemp (Cannabis sativa). In one embodiment vegetable species, especially Solanum species (including Lycopersicon species) are preferred.

Thus, for example species of the following genera may be transformed: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Cucumis, Hyoscyamus, Lycopersicon, Solanum, Nicotiana, Malus, Petunia, Digitalis, Majorana, Ciahorium, Helianthus, Lactuca, Bromus, Citrullus, Asparagus, Antirrhinum, Heterocallis, Nemesis, Pelargonium, Panieum, Pennisetum, Ranunculus, Senecio, Salpiglossis, Browaalia, Glycine, Pisum, Phaseolus, Gossypium, Glycine, Lolium, Festuca, Agrostis.

In one embodiment the ratio of the virE2 mutant (comprising the selectable marker gene) to the virE2 donor strain (comprising the GOI) is about 1:3. Other suitable ratios include any ratio wherein preferably more donor strain is used relative to the virE2 mutant strain, for example ratios above 1:1, such as 1:2, 1:4, 1:5, or others. However, e.g. for tobacco, a ratio of 1:1 was found to be more suitable than ratios above 1:1.

The two strains are preferably freshly grown. The strains may be mixed prior to contact with the plant or plant tissue or may be brought in contact with the cells in separate steps, e.g. consecutively or simultaneously.

Amounts of each strain include for example an OD at 600 nm in liquid MS20 medium between about 0.100 and about 0.300, or an equivalent amount.

In step (e) plant cells, tissues, organs or plants (or plantlets) and/or the regenerated plants or plantlets are subjected to a selection step, based on the marker gene and its expression product. Step (e), therefore, comprises the step of selecting plant cells and/or regenerated plants (or plantlets) using the phenotype conferred by the selectable marker gene product. The purpose of this step is to select the co-transformants.

In this step standard methods may be used. For example roots and/or shoots may be regenerated and (part of) the regenerated tissue may be screened and selected based on the marker gene and/or its expression product or phenotype conferred thereby. As already mentioned above, methods for regenerating whole plants from transformed plant cells are known in the art and can be applied to the transformed and/or selected cells. Depending on the type of marker genes used, selection methods may be different. If the marker gene is a herbicide resistance gene, plantlets or parts of leaves may be contacted with the herbicide and those plantlets which are resistant to the herbicide are then selected. The selected plants may be primary transformants (T1 generation), but selection may alternatively or in addition also take place at a later stage, e.g. in later generations obtained by selfing and/or crossing. Thus, optionally the plants may further be crossed and/or selfed to produce whole plants and offspring comprising the marker gene DNA in the genome.

Due to the high efficiency of the instant co-transformation a large percentage of the primary tranformants containing the marker gene also contain the GOI. Thus, more than 50%, such as at least 60%, 70%, 80%, 90% or more (95%, 99%, 100%) of selected transformants also contain the GOI in addition to the marker gene. The non selected plants may be discarded. The higher the transformation efficiency, the better, as fewer transformants need to be analyzed subsequently for the presence of the GOI, using laborious molecular methods, such as PCR for the GOI or nucleic acid hybridizations (e.g. Southern blotting analysis) for the GOI. In the Examples herein 100% of the selected plants contained both T-DNAs, as confirmed by PCR analysis. Thus, much fewer total numbers of transformants are required compared to traditional co-transformation methods.

The method then optionally further comprises step (f): crossing and/or selling the selected plants (or plants derived from the selected plant cells or plantlets) to produce offspring, and (g) optionally discarding those offspring which comprise the selectable marker gene and retaining those offspring which comprise the gene of interest but lack the selectable marker gene (i.e. segregating away the marker gene from the gene of interest to produce marker free plants comprising the gene of interest).

As the marker gene and GOI will be able to segregate from each other, optionally those plants or offspring which comprise the selectable marker gene may be discarded and those plants or offspring which comprise the GOI, but which lack the selectable marker gene may be retained for further use. Thus, plants comprising only the GOI integrated in the genome (marker free plants) may be generated, from which the marker gene has been segregated away. Selection of such plants can be done using known methods, e.g. PCR screening, hybridization based methods and/or using phenotypic screens.

### Agrobacterium Strains and Ti-plasmids According to the Invention

It is also an embodiment of the invention to provide Agrobacterium strains and pairs of Agrobacterium strains (at least one virE2 donor strain and at least one virE2 mutant strain) for use in the above method.

The virE2 donor strain preferably further comprises a T-DNA which comprises a GOI, within the cell (e.g. on a plasmid), while the virE mutant strain preferably further comprises a T-DNA which comprises a marker gene within the cell (e.g. on a plasmid).

In one embodiment the virE2 mutant strain comprises a Ti-plasmid having a mutation in the virE2 gene, preferably a DNA insertion, resulting in no functional virE2 protein being made by the strain. In a preferred embodiment the virE2 mutant strain is the strain deposited under Accession number CBS 121809, or a derivative thereof, which retains the Ti-plasmid found therein. In another embodiment a strain comprising the Ti-plasmid of CBS121809 is provided, as is a strain comprising the mutant virE2 gene found in the Ti-plasmid of strain CBS121809.

### Uses According to the Invention

The uses are already clear from the description herein above. In one embodiment the use of a first Agrobacterium strain incapable of producing a functional virE2 protein and comprising a T-DNA comprising a selectable marker gene together with a second Agrbobacterium strain comprising a gene encoding a functional virE2 protein and a T-DNA comprising a gene of interest for co-transformation of a plant, plant cell or plant tissue with said two T-DNAs is provided herein.

The virE2 gene of the first Agrobacterium strain preferably comprising an insertion, deletion or replacement of all or part of the virE2 gene.

### Kits According to the Invention

Also a co-transformation kit is provided, which comprises a first and a second Agrobacterium strain, the first strain being incapable of producing a functional virE2 protein due to an insertion, deletion or replacement of all or part of the virE2 gene of the Ti-plasmid present in the strain and the second strain comprising a gene encoding a functional virE2 protein.

The second strain preferably comprises at least two virE2 genes, encoding functional virE2 protein, as described.

The strains may be supplied in frozen or freeze dried viable cultures or as live cultures, e.g. on plates. Also, the strain may be provided separately or as mixtures, e.g. in appropriate ratios and/or concentrations for use in co-transformation. Other materials may be provided in the kits, such as protocols, controls, virE2 DNA (e.g. probes and/or primers), buffers, and the like.

Also, the transformed and/or regenerated transgenic plant cells and plants (and/or plant parts) produced using the above method are encompassed herein.

### Sequences

SEQ ID NO 1: virE2 cDNA
SEQ ID NO 2: virE2 protein encoded by SEQ ID NO: 1.
SEQ ID NO 3: virE operon comprising a (knock-out) mutant virE2 cDNA, due to an insertion
SEQ ID NO 4: virG protein (X62885)
SEQ ID NO 5: virE1 protein (AA250537)

### FIGURE LEGENDS

FIG. 1—map of plasmid pKG6305

FIG. 2—a map of plasmid pKG6330

FIG. 3—overview of T-DNA transfer from Agrobacterium (bottom) into a plant cell (top)

FIG. 4A—co-transformation using non-mutant strains, whereby the cotransformation efficiency is less than 50% (the black dots indicate virE2 protein)

FIG. 4B—method according to the invention, leading to a high co-transformation efficiency (the black dots indicate virE2 protein)

The following non-limiting Examples describe the co-transformation method and strains according to the invention. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, and Sambrook and Russell (2001) Molecular Cloning: A Laboratory Manual, Third Edition, Cold Spring Harbor Laboratory Press, NY; and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

### EXAMPLES

### Example 1

### Construction and Confirmation of EHA105-dE2

Agrobacterium tumefaciens strain EHA105-dE2 (“d” stands for disrupted) was made by integrating a disruption plasmid (named pKG6328) incapable of replicating in Agrobacterium tumefaciens into the EHA105 VirE2 gene through homologous recombination. The Ti plasmid of EHA105 is a derivative of Ti plasmid pTiBo542.

Insertion of pKG6328 into the VirE2 open reading frame (ORF) was confirmed by amplifying the border parts of pKG6328 into the Ti plasmid and sequencing them. For testing (dis)functionality a transformation experiment was performed, including as a control a complementation of the knock-out by adding a plasmid capable of replicating in Agrobacterium tumefaciens and expressing both the pTiBo542 VirE1 and VirE2 genes, driven by the VirE operon promoter (plasmid pKG6330).

Strain EHA105-dE2 has been deposited by the applicant under the Budapest Treaty at the “Centraalbureau voor Schimmelcultures” (CBS, P.O. Box 85167, 3508 AD Utrecht, The Netherlands) or “Fungal Biodiversity Centre” in Utrecht, The Netherlands, under Accession number CBS121809 on 30 Aug. 2007.

Construction of Disruption Vector pKG6328

Part of the pTiBo542 ORF was amplified directly on EHA105 bacteria with VirE2 gene specific primers including a specific part with stop codons in three reading frames (Fw: 5′-TGAATGAATGATGATGACACTGAC-3′ and Rev: 5′-TTAGTCAATTAGTCGCCGGCAAACCTGT-3′).

The following PCR profile was used: 3 minutes 80° C.-2 minutes 94° C.-(30 seconds 94° C.-1 minute 55° C.-1 minute 72° C.) 30 times-4° C. forever. The resulting PCR product was ligated into a PCR cloning vector from Invitrogen using the R6K origin of replication. The resulting plasmid was sequenced to confirm incorporation of the stop codons at each side of the part of the VirE2 ORF. This plasmid was called pKG6328.

Disruption of Agrobacterium tumefaciens Strain EHA105 VirE2 gene

Plasmid pKG6328 was electroporated to competent cells made from EHA105. Since this plasmid is not capable of replicating inside Agrobacterium tumefaciens, it needs to integrate by homologous recombination using the VirE2 ORF part, disrupting the endogenous VirE2 gene in the process. Transformants were selected on kanamycin, a selection marker present on the pKG6328 vector backbone. Disruption of the VirE2 gene was confirmed by amplifying the integration sites and sequencing them (SEQ ID NO: 3). This new strain was named EHA105-dE2 and was deposited as CBS121809.

Construction of pKG6305

Plasmid pBBR1MCS (GENBANK NR. UO2374, Kovach et al. 1994, BioTechniques 16 (5), 800-802) is the backbone vector for both pKG6305 and pKG6330. The VirG gene was amplified using the following primers: Reverse: 5′-CTCGCGTCATTCTTTGCTGGA-G3′ and Forward: 5′-TCCGGGATCGATTTCAACAATAC-3′. As a template 50 ng total DNA from A. tumefaciens strain EHA105 was used with the following PCR profile: 2 min 94° C.-(30 sec 94° C.-1 min 58° C.-2 min 68° C.)×30-10 min 72° C.-4° C. forever. A proofreading DNA polymerase was used to reduce the risk of PCR errors (rTth polymerase from Perkin Elmer). Using AmpliTaq red from Perkin Elmer an “A-overhang” was created by adding 1 unit of this polymerase to the PCR reaction and incubating for 10 minutes at 72° C. This PCR product was ligated into the pCR2.1 PCR cloning vector from Invitrogen and sequenced.

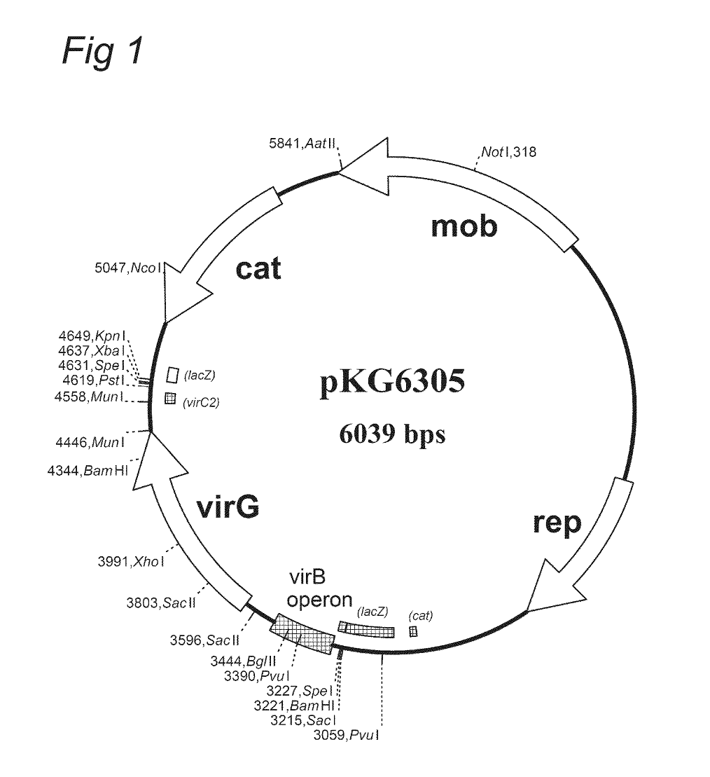

From this vector the VirG gene was obtained by cutting the plasmid with PstI and Sad and isolating the 1393 bps fragment with the gene from gel. This fragment was ligated into the corresponding sites of pBBR1MCS. This new plasmid was given a new Multiple Cloning Site (MSC) for cloning steps later on. This MCS consisted of 2 oligos: 5′-TTCTAGAACTAGTGGGCCCCTGCAGGTTAACATGCA-3′ and 5′-TGTTAACCTGCAGGGGCCCACTAGTTCTAGAAGGCC-3′. When these two oligos form a double stranded piece of DNA after they have been phosphorylated, it will have single stranded DNA overhangs that fit on overhangs created by ApaI and PstI. Using these two enzymes the adapter was ligated into these sites. This end product is pKG6305. In FIG. 1 a map of plasmid pKG6305 can be found.

Construction of pKG6330

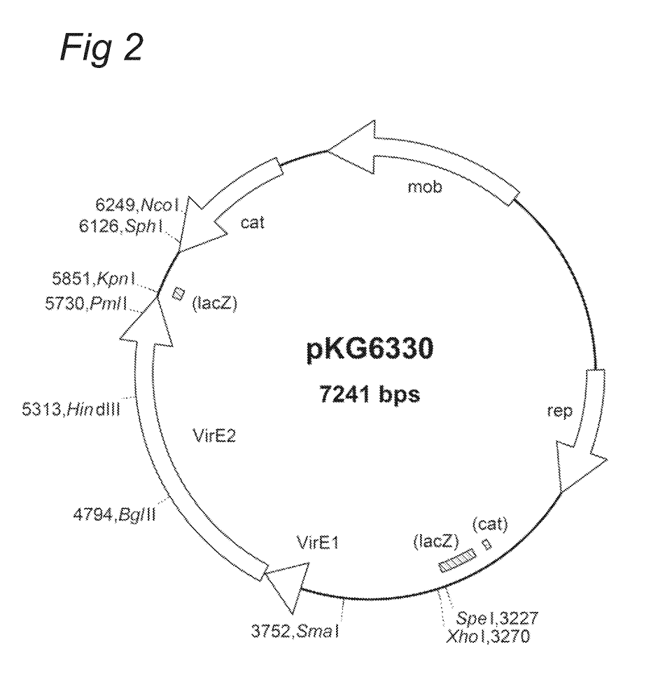

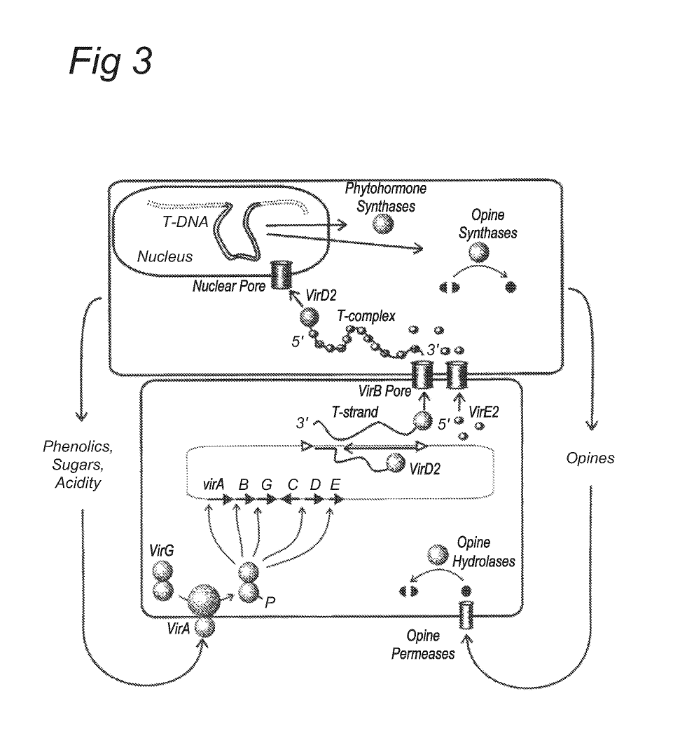

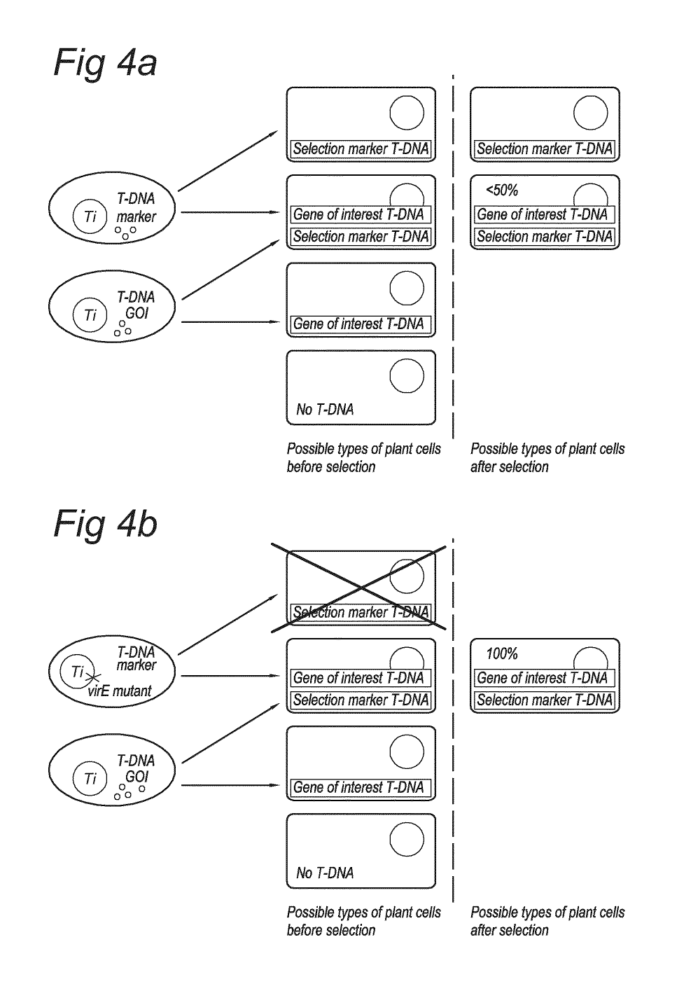

The VirE operon promoter, the VirE1 gene, and the VirE2 gene were amplified from EHA105 using gene specific primers and cloned into a plasmid backbone carrying the pBBR origin of replication and chloramphenicol resistance gene (for selection in bacteria). To amplify this VirE operon part the following primers were used: Fw: 5′-ACTAGTTGCCCGCGAAACAGCATTGACT-3′ and Rv: 5′-GGGTACCATGACGCGGCAGCAGGAAC-3′. With the forward primer a SpeI restriction enzyme site was built in, with the reverse primer a KpnI site. Sites have been underlined in the primer sequences. As a template 50 ng total DNA from A. tumefaciens strain EHA105 was used with the following PCR profile: 2 min 94° C.-(30 sec 94° C.-1 min 58° C.-3 min 68° C.)×30-10 min 72° C.-4° C. forever. A proofreading DNA polymerase was used to reduce the risk of PCR errors (rTth polymerase from Perkin Elmer). The PCR product was subsequently cut with SpeI and KpnI and ligated into the corresponding sites of pKG6305. This plasmid can replicate both in Escherichia coli and Agrobacterium tumefaciens. After sequence verification this plasmid has been transferred to Agrobacterium by electroporation. In FIG. 2 a map of plasmid pKG6330 can be found.

### Virulence and Complementation Tests

When using EHA105-dE2 supplemented with a T-DNA for gusA expression for plant transformation, the resulting number of transformed cells should be close to zero because of the disruption of the VirE2 gene. Also, when adding an intact copy of the VirE operon to this strain, the phenotype should be complemented and the virulence restored to levels of a strain with a non disrupted VirE2 (EHA105 without disruption of the VirE2 gene). Nicotiana benthamiana transformations have been performed (both leaf disc transformations and Agrobacterium-infiltrations) to evaluate the virulence of EHA105-dE2. The following strain-plasmid combinations have been used:

1. EHA105 with T-DNA (positive control);
2. EHA105-dE2 with T-DNA (should be close to zero);
3. EHA105-dE2 with a T-DNA, and pKG6330 (complementation test).

For leaf disc transformations 1 cm diameter leaf discs were cut from Nicotiana leafs. The protocol used for tobacco leaf disc transformation has been described in Horsch et al (1988, Plant molecular biology manual A5:1-9). For each Agrobacterium treatment 40 leaf discs were transformed. After 10 days after the start of the co-cultivation, leaf discs were used for GUS staining using Xgluc as a substrate (Jefferson et al. 1987 EMBO J. December 20; 6(13): 3901-3907). For each leaf disc the number of blue spots was counted. The results of the leaf disc transformations are given in Table 1 below.

TABLE 1
The average (AVG) number of gus spots per leaf disc obtained after
transformation using different strain - plasmid combinations.

Number of gus spots per leaf disc

| Strain | AVG | SD | MAX | MIN |
|---|---|---|---|---|
| EHA105 with T-DNA | 49 | 15.6 | 73 | 12 |
| EHA105-dE2 with | 0.4 | 0.6 | 2 | 0 |
| T-DNA | | | | |
| EHA105-dE2 with | 39 | 12 | 61 | 19 |
| T-DNA, and | | | | |
| pKG6330 | | | | |

For each the standard deviation (SD), the maximum number (MAX) and minimum number (MIN) have been calculated as well.

Both the leaf disc transformations and the Agrobacterium infiltrations (data not shown; show that EHA105-dE2 is not capable of any significant transfer of T-DNA to plant cells, as a consequence of the VirE disruption. The insertion in VirE2 however, can be complemented by adding a separate plasmid expressing VirE2.

### Example 2

### Co-transformation Using EHA105-dE2

In the following co-transformation experiments strain EHA105-dE2 is used as the strain to deliver the T-DNA with the nptII gene (for plant transformant selection, giving kanamycin resistance; the nptII gene exemplifies the plant selectable marker gene according to the invention) and EHA105 for delivering the T-DNA with the gusA gene (the gusA gene exemplifies the gene-of-interest, GOI, according to the invention). To compare the performance of this couple, other combinations have been used as well.

### Used Plasmids

Plasmid pKG6330 carrying (part of) the pTiBo542 VirE operon has been described in Example 1 and FIG. 2. Plasmid pKG6305 has the same vector backbone as pKG6330, but carries the pTiBo542 VirG gene promoter and VirG gene ORF and is also described in Example 1 and FIG. 1. Furthermore two plasmids with a T-DNA have been used. One plasmid carries a nos promoter—nptII gene—nos terminator construct between the Agrobacterium RB and LB sequences, the other carries a ³⁵S promoter—gusA gene with potato LS1 intron—nos terminator construct between the Agrobacterium RB and LB sequences (the gusA gene with LS1 intron has been described by Vancanneyt et al 1990, MGG 220(2):245-250). The strain with the nptII T-DNA will be referred to as “nptII donor”, the strain with the gusA T-DNA will be referred to as “gusA donor”.

### Strain—Plasmid Combinations Used for Co-Transformation

Below in Table 2 the tested strain-plasmid combinations are shown. Additional to these combinations in each experiment a non-transformed control on both selection medium and medium without selection was included as well.

TABLE 2
Combinations of Agrobacterium tumefaciens strains and
plasmids used for (co-)transformation experiments.

| nptII donor (marker | | |
|---|---|---|
| gene) | gusA donor (GOI) | Remark |
| EHA105 | None | transformation control |
| EHA105 | EHA105 | reference combination 1 |
| EHA105 | EHA105 + pKG6305 | reference combination 2 |
| EHA105 | EHA105 + pKG6330 | reference combination 3 |
| EHA105-dE2 | None | negative control |
| EHA105-dE2 | EHA105 | test combination 1 |
| EHA105-dE2 | EHA105 + pKG6305 | test combination 2 |
| EHA105-dE2 | EHA105 + pKG6330 | test combination 3 |

### Tomato Cotyledon Explant Transformation Experiments

The tomato transformation protocol has been described in Koornneef et al. (Transformation of tomato; In: Tomato Biotechnology, Donald Nevins and Richard Jones, eds. Alan Liss Inc., New York, USA, pag. 169-178) and Koornneef et al. (1987, Theor. Appl. Genet. 74: 633-641). For co-transformation the diluted Agrobacterium cultures are mixed in a ratio of 1:3 for nptII donor strain and gusA donor strain respectively. The remainder of the described protocol has been unchanged. When tomato shoots appeared, they are harvested and rooted on solid MS20 medium containing 1 mg/l IBA, 200 mg/l cefotaxime, 200 mg/l vancomycin, and 100 mg/l kanamycin.

Analysis of Obtained Plantlets

DNA is isolated from the regenerated plantlets and analyzed using PCR for the presence of the T-DNAs. For nptII the PCR primers are as follows: Fw 5′-GTCCCGCTCAGAAGAACT-3′ and Rv 5′-GGCACAACAGACAATCGG-3′; the gusA primer pair is as follows: Fw 5′-GGGCAGGCCAGCGTATCGT-3′ and Rv 5′-GTGTTCGGCGTGGTGTAGAGCAT-3′. For each reaction 50 ng of plant DNA and 1 unit of AmpliTaq red (Perkin Elmer) is used. The PCR profile to be used is: 3 min 94° C.-(30 sec 94° C.-30 sec 55° C.-1 min 72° C.)×30-4° C. forever. On a 1.5% agarose gel in 1×TAE stained with Ethidiumbromide (EtBr) 10 μl of each reaction will be analyzed.

To evaluate the way both T-DNAs have integrated in the genome (in a coupled or a non-coupled manner) selfings were made of the original transformants. The progeny was analyzed using PCR for the presence of both T-DNAs as described above.

### Results

From the treatment EHA105-dE2 (nptII donor) with EHA105 (gusA donor), Table 2, test combination 1, five plants were regenerated. Using PCR as described above, PCR products indicating the presence of both T-DNAs were obtained from all five plants (100% co-transformation efficiency).

### Example 3

### Co-Transformation of Tobacco

A second co-transformation experiment was performed on tobacco (Nicotiana tabacum) cv. SR1 using Agrobacterium transformation and regeneration and GUS staining protocols well known in the art. The co-transformation treatments applied consisted of a mixture of the VirE2-deficient strain carrying a nptII T-DNA with the wild-type strain carrying a gus T-DNA only, in a ratio of 1:3 or alternatively in a ratio of 1:1. For both treatments, control conditions were also applied, consisting of only non-deficient (VirE2 wild-type) strains in the same mixing ratios. The results are summarized in Table 3. In the best conditions (mix ratio of 1:1), a co-transformation efficiency of 82.5% was observed (treatment 3), versus a co-transformation efficiency with control strains of 53.3% (treatment 4). This effect is significant at P≦0.05 (Chi-square=25.82). When a mix ratio of the two strains of 1:3 was applied (treatment 1), the co-transformation efficiency was 74.5% versus 55.6% in the control. This difference was likewise significant at P≦0.05 (Chi-square=6.81).

TABLE 3
The average (AVG) number of GUS positive plants obtained after
transformation of tobacco with a VirE2-deficient strain.

| | Kanamycin-resistant plants | | | |
|---|---|---|---|---|
| | | | | % |
| Treatment | # plants | GUS+ | GUS− | cotransf. |
| EHA105 with T-DNA nptII | 28 | 0 | 28 | n.a. |
| EHA105-dE2 with T-DNA nptII | 11 | 0 | 11 | n.a. |
| Cotransformation treatment 1 | 47 | 35 | 12 | 74.5 |
| Cotransformation treatment 2 | 18 | 10 | 8 | 55.6 |
| Cotransformation treatment 3 | 40 | 33 | 7 | 82.5 |
| Cotransformation treatment 4 | 15 | 8 | 7 | 53.3 |

Cotransformant treatment 1 is a 1:3 mixture of Agrobacterium straim EHA105-dE2 with nptII T-DNA:EHA105 with gus T-DNA.
Cotransformant treatment 2 is a 1:3 mixture of Agrobacterium straim EHA105 with nptII T-DNA:EHA105 with gus T-DNA.
Cotransformant treatment 3 is a 1:1 mixture of Agrobacterium straim EHA105-dE2 with nptII T-DNA:EHA105 with gus T-DNA.
Cotransformant treatment 4 is a 1:1 mixture of Agrobacterium straim EHA105 with nptII T-DNA:EHA105 with gus T-DNA.

1. A method for making a selectable marker gene-free transgenic plant comprising a gene of interest, said method comprising the steps of:
(a) providing bacteria of two Agrobacterium strains,
(i) a virE2 donor strain comprising a gene encoding a functional virE2 protein and
(ii) a virE2 mutant strain that cannot produce functional virE2 protein,
(b) introducing a T-DNA comprising a gene-of-interest into the virE2 donor strain bacteria,
(c) introducing a T-DNA comprising a selectable marker gene into the virE2 mutant strain bacteria,
(d) exposing plant cells to bacteria of both of said virE2 strains,
(e) selecting plant cells or regenerated plants by selecting for the phenotype conferred by the selectable marker gene
(f) optionally, crossing or selfing the selected plants to produce offspring, and
(g) optionally, discarding those offspring which comprise the selectable marker gene and retaining those offspring which comprise the gene of interest but lack the selectable marker gene.

2. The method according to claim 1, wherein the virE2 donor strain comprises at least two virE2 genes, each encoding a functional virE2 protein.

3. The method according to claim 1, wherein, in the virE2 mutant strain,
(A) the virE2 gene comprises an insertion, and/or
(B) part or all of the virE2 gene is deleted and/or replaced.

4. The method according to claim 3, wherein the virE2 mutant strain is deposited at Centraalbureau voor Schimmelcultures under Accession number CBS 121809, or a derivative thereof.

5. The method according to claim 2, wherein one of said at least two virE2 genes is on a Ti-plasmid and at least one of said virE2 genes is on a helper plasmid.

6. The method according to claim 1, wherein said T-DNAs are introduced as a DNA vector or plasmid and wherein the T-DNAs comprise at least a right border sequence.

7. The method according to claim 1 wherein the ratio of the virE2 mutant strain bacteria to the virE2 donor strain bacteria is selected from the group consisting of 1, 2, 3, 4, 5 and >5.

8. The method according to claim 1, wherein co-transformation efficiency of said plant cells is at least 60%.

9. An Agrobacterium strain comprising a DNA insertion in the virE2 gene, which results in an inability of bacteria of the strain to make functional virE2 protein.

10. The Agrobacterium strain according to claim 9 deposited at Centraalbureau voor Schimmelcultures under Accession number CBS121809, or a derivative thereof.

11. A method for co-transforming a plant cell with two T-DNAs, comprising exposing the plant cell to:
(a) bacteria of a mutant Agrobacterium strain that cannot produce functional virE2 protein and which comprise a T-DNA comprising a selectable marker gene; and
(b) bacteria of an Agrobacterium strain that comprise
(i) a gene encoding a functional virE2 protein, and
(ii) a T-DNA comprising a gene of interest,
thereby co-transforming said plant cell.

12. The method according to claim 11, wherein the virE2 mutant strain bacteria comprise a virE2 gene characterized by an insertion, and/or a partial or complete deletion or replacement of the gene.

13. A co-transformation kit comprising bacteria of a first and a second Agrobacterium strain, each strain comprising a Ti plasmid, wherein
(a) bacteria of the first strain cannot produce a functional virE2 protein due to an insertion in, deletion from and/or replacement of the virE2 gene of the Ti-plasmid, and
(b) bacteria of the second strain comprise a gene encoding a functional virE2 protein.

14. The kit according to claim 13, wherein the bacteria of the second strain comprise at least two virE2 genes each encoding a functional virE2 protein.

15. The method according to claim 8, wherein the co-transformation efficiency of said plant cells is at least 80%.

16. The method according to claim 8, wherein the co-transformation efficiency of said plant cells is at least 90%.

17. The method according to claim 8, wherein the co-transformation efficiency of said plant cells is 100%.


SEQUENCE LISTING (5 sequences)

SEQ ID NO: 1 - DNA, 1650 bp, Agrobacterium tumefaciens
atggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttga

SEQ ID NO: 2 - protein, 549 aa, Agrobacterium tumefaciens
MDPSSNENVYVGRGHNIENDDDTDPRRWKKANISSNTISDIQMTNGEDVQSGSPTRTEVVSPRLDYGSVDSSSSLYSGSEHGNQAEIQKELSVLFSNMSLPGNDRRPDEYILVHQTGQDAFTGIAKGNLDQMPTKAEFNACCRLYRDGAGNYYPPPLAFDKISVPEQLEEKWGMMEAKERNKLRFQYKLDVWNHAHADMGITGTEIFYQTDKNIKLDRNYKLRPEDRYVQTEKYGRREIQKRYQHELQAGSLLPDIMIKTPQNDIHFVYRFAGDNYANKQFSEFEHTVKRRYGDETEIKLKSKSGIMHDSKYLESWERGSADIRFAEFVGENRAHNRQFPTATVNMGQQPDGQGGLTRDRHVSVDFLMQSAPNSPWAQALKKGELWDRVQLLARDGNRYLSPPRLEYSDPAHFTELMNRVGLPASMGRQSHAASIKFEKFDAQAAVIVLNGPELRDIHDLSPEKLQNLSTKDVIVADRNENGQRTGTYTSVAEYERLQLRLPPDAAGVLGEATDKYSRDFVRPEPASRPISDSRRIYESRPRSQSVNSF

SEQ ID NO: 3 - DNA, 5662 bp, Agrobacterium tumefaciens
  virE2 (203)..(1024)
  stop (1025)..(1035)
  loxP (1057)..(1090)
  R6Kori (1111)..(1502)
  nptII promoter (1696)..(1833)
  nptII gene (1863)..(2657)
  3′ end T7 (2807)..(2935)
  3′ end bgh (2950)..(3158)
  6xhis (3181)..(3198)
  v5 epitope (3199)..(3240)
  stop (3289)..(3299)
  virE2 (3295)..(4890)
atggccatcatcaagccgcatgtgaacaaaaataggacaacctcgccgatagagagaccggagtctctcatagaggaaatgagcggcagtcatccgccgagtggttttaccaacctggatctcgctatgatcgagctggaggactttgtccatcggtgcccgctcccagaagacaatcttgctggtcagaaggagtgagacgatggatccgtctagcaatgagaatgtctatgtgggtcgcggtcacaacatcgaaaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgactaattgactaaccggtcgagccaattcctccgataacttcgtataatgtatgctatacgaagttatggtaccgcggccgcgtagaggatctgttgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagcttttaaggtttaacggttgtggacaacaagccagggatgtaacgcactgagaagcccttagagcctctcaaagcaattttgagtgacacaggaacacttaacggctgacatgggaattagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgggatatccggatatagttcctcctttcagcaaaaaacccctcaagacccgtttagaggccccaaggggttatgctagttattgctcagcggtggcagcagccaactcagcttcctttcgggctttgttagcagccggatcttctagaatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatagcgagcttcaatggtgatggtgatgatggctagaatcgagaccgaggagagggttagggataggcttaccgagctcccgaattgccctttgaatgaatgatgatgacactgaccccaggcgttggaagaaggcgaatatcagttccaacaccatctccgatattcagatgacgaatggcgaagacgtacaatcagggagccctacccgaacggaagttgtaagcccacgtctggattatggatcggtcgactcctcctccagcctttattctggcagcgagcacggaaatcaagctgagattcaaaaagagctgtccgtcttgttctcgaacatgtctttgccaggcaacgatcggcgcccggacgaatacattctcgtgcatcaaacgggacaagatgcttttactggtattgccaaaggcaacctcgaccaaatgcccaccaaggcggaatttaacgcgtgctgccgtctctacagggacggagccggtaattactacccgccacctctcgcattcgacaagattagcgttccagagcaactggaggaaaaatgggggatgatggaggcgaaggaacgtaacaaactgcggtttcagtacaagttggacgtatggaatcatgcgcacgctgatatggggatcacgggcacagagatcttttatcaaacagataagaacataaagctcgaccggaattataaactaagacctgaagaccgatacgtacaaacagaaaaatacgggcgccgggaaattcaaaagcgatatcaacacgaactccaggctggttcgctgctgcccgatattatgatcaaaactccccaaaatgacatccacttcgtgtacaggtttgccggcgacaattacgccaacaaacagttcagcgagtttgaacacaccgtcaagcgcaggtatggcgacgagactgagatcaaattgaagtcaaagtcaggcattatgcatgactcgaaatatctggaatcctgggaacggggcagtgcggatattcgcttcgcggaattcgttggggaaaatagagctcacaatcggcagtttccaactgcgacagtaaatatgggacagcagccagacgggcagggcggtttgacccgcgaccgtcatgtgagcgttgacttcctaatgcaaagcgcacccaattcgccttgggcgcaagctttgaaaaagggagaactgtgggatcgcgttcagttgcttgctcgcgacggcaaccgctatctgtcgccgcccagattggaatattctgaccctgcacatttcaccgagttgatgaaccgggttggtttacccgcatcgatgggtcggcaaagccatgcggctagtatcaaattcgaaaagtttgacgcgcaggcagcggttattgtcttaaatggcccagagttacgtgacattcatgacttgtctcctgaaaaactgcaaaatttgtccaccaaagatgtcatcgtcgccgatcgcaatgagaatggtcagagaactggcacgtacaccagcgtcgcggaatatgagcgcttgcagttaaggctgccacccgatgcagcgggggtgcttggtgaagcaactgacaaatattcacgtgatttcgttcggccagagccggcgtcgcgtccaatcagtgacagccgcaggatatacgaaagtcgaccgcgtagccaaagcgtcaacagcttttgacgttcctgctgccgcgtcaacgaggaagctcgtttgacccgggtttgccaatgaaagggctcaatcatggtgaacactacaaagaaaagttttgcgaagtcgcttacggcagatatgcgccgttctgctcagcgcgttgtcgagcaaatgygaaamgcattgattaccgaagaagaggcgctcaagcggcaagccagactggagagtcccgataggaagcgaaagtatgctgctgatatggcgatagtcgacaaactcgacgtagggtttcgaggcgaaataggctataaaattcttggaaataaccggcttcgagtagacaaccataaagaattaacgcgtgagcacggtagacttcgcaaaaccaaaacggttctgaagcgtaacccggtgacgcaggaagtctatttgggtttatatgaaaggaagtcctggttaagtgtcagcagccatttgtatgctgcggacggcacactccgcatgaagcacgtgaaatacaaagacggacgttttgaggaaaaatgggagcgcgacgaaaatggcgacctgatccgcacaaggtacgccaaccgtggcaggctctttcaacctgtatccgagaaaatgggcgcgccgtatcggagcggccctgacgaccggctctatcgcgatctaacccgtcgaaacggtttcagacgggagacattcgaacgggacgatcacggaaacctcgagcgtatcggcagcaaccatgtcggcttttccaagatttcagtgaaggcagccaatcgtc

SEQ ID NO: 4 - protein, 267 aa, Agrobacterium tumefaciens
MIVHPSRENFSSAVNKGSDFRLKGEPLKHVLLIDDDVAMRHLIIEYLTIHAFKVTAVADSTQFTRVLSSATVDVVVVDLNLGREDGLEIVRNLAAKSDIPIIIISGDRLEETDKVVALELGASDFIAKPFSTREFLARIRVALRVRPNVVRSKDRRSFCFTDWTLNLRQRRLMSEAGGEVKLTAGEFNLLLAFLEKPRDVLSREQLLIASRVRDEEVYDRSIDVLILRLRRKLEADPSSPQLIKTARGAGYFFDADVQVSHGGTMAA

SEQ ID NO: 5 - protein, 65 aa, Agrobacterium tumefaciens
MAIIKPHVNKNRTTSPIERPESLIEEMSGSHPPSGFTNLDLAMIELEDFVHRCPLPEDNLAGQKE